SEQUENCE LISTING (14 sequences)

SEQ ID NO: 1 - DNA, 50 bp, Artificial Sequence
  LK01 primer
gtgagtgagtggccgacggggccgctggaatggacaacaatcccaacatc

SEQ ID NO: 2 - DNA, 54 bp, Artificial Sequence
  LK02 primer
tgagtgagtcggccccagaggccctgcagctccctcgagcgttgcagtaacggg

SEQ ID NO: 3 - DNA, 20 bp, Artificial Sequence
  LK04
gagcctcgaaaggtaccgtc

SEQ ID NO: 4 - DNA, 20 bp, Artificial Sequence
  LK03 primer
gacggtacctttcgaggctc

SEQ ID NO: 5 - DNA, 90 bp, Artificial Sequence
  PPELB primer
ctcgctcgcccatatgcggccgcaggtctcctcctcttagcagcacaaccagcaatggccatggataacaatccgaacatcaatgaatgc

SEQ ID NO: 6 - DNA, 50 bp, Artificial Sequence
  PCRY1 primer
atccgataaatagctagctaaattggacacttgatcaatatgataatccg

SEQ ID NO: 7 - protein, 7 aa, Artificial Sequence
  C-terminal sequence of synthetic Cry1Ac from Figure 1
MDNNPNI

SEQ ID NO: 8 - protein, 12 aa, Artificial Sequence
  N-terminal sequence of synthetic Cry1Ac from Figure 1
RFEFIPVTATLE

SEQ ID NO: 9 - protein, 15 aa, Artificial Sequence
  C-terminal sequence of Cry1Ac-fUSE5 and Cry1Ac-Kpn-fUSE5 from Figure 1
HSADGPLAMDNNPNI

SEQ ID NO: 10 - protein, 26 aa, Artificial Sequence
  N-terminal sequence of Cry1Ac-fUSE5 and Cry1Ac-Kpn-fUSE5 from Figure 1
RFEFIPVTNARGSCRASGAETVESCL

SEQ ID NO: 11 - protein, 37 aa, Artificial Sequence
  Protein sequence of WT-fUSE5 from Figure 1
VKKLLFAIPLVVPFYSHSADVAMGWAAAGAETVESCL

SEQ ID NO: 12 - protein, 20 aa, Artificial Sequence
  C-terminal sequence of Cry1Ac-SZ from Figure 1
AGLLLLAAQPAMAMDNNPNI

SEQ ID NO: 13 - protein, 12 aa, Artificial Sequence
  Internal sequence of Cry1Ac-SZ from Figure 1
RFEFIPVTATLE

SEQ ID NO: 14 - protein, 20 aa, Artificial Sequence
  N-terminal sequence of Cry1Ac-SZ from Figure 1
VSNLASGGGGSPFVCEYQGQ

(19) United States
(12) Patent Application Publication
(10) Pub. No.: US 2004/0254364 A1
(43) Pub. Date: Dec. 16, 2004

(54) Phage display of a biologically active Bacillus thuringiensis toxin

(76) Inventors: Michael J. Adang, Athens, GA (US); Laura M. Kasman, Mt. Pleasant, SC (US)

Correspondence Address: SALIWANCHIK LLOYD & SALIWANCHIK, A PROFESSIONAL ASSOCIATION, PO BOX 142950, GAINESVILLE, FL, 32614-2950, US

(21) Appl. No.: 10/828,919

(22) Filed: Apr. 20, 2004

Related U.S. Application Data
(63) Continuation of application No. 09/629,596, filed on Jul. 31, 2000.
(60) Provisional application No. 60/146,646, filed on Jul. 30, 1999.

(51) Int. Cl. C12Q 1/68; C07H 21/04; C12N 5/10; C12N 15/74

(52) U.S. Cl. 536/23.71; 435/471; 435/69.1; 435/6; 435/419

(57) ABSTRACT

Disclosed herein are activated Bt toxins expressed in E. coli as a translational fusion with a phage coat protein of filamentous phage. Phage displaying this fusion protein were viable, infectious, and as lethal as pure toxin on a molar basis when fed to insects susceptible to native Bt toxins.

### CROSS-REFERENCE TO RELATED APPLICATIONS

The present application is a continuation application of U.S. application Ser. No. 09/629,596; filed Jul. 31, 2000 and claims the benefit of U.S. Provisional Application Ser. No. 60/146,646; filed Jul. 30, 1999, which is hereby incorporated by reference in its entirety.

The subject invention was made with government support under a research project supported by NIH (No. AI29092) and USDA/NR1 (Nos. 95-37302-1803 and 95-37302-4548).

### BACKGROUND OF THE INVENTION

The crystal proteins of Bacillus thuringiensis (Bt) are remarkably potent and species-specific biological pesticides. Effective when ingested at sub-picomolar doses, Bt preparations consisting of killed bacteria have been an important alternative to chemical pesticides for over 3 decades, although their relatively short shelf life and poor persistence in the field have limited their use (Adang, M. J. (1991), Feitelson, J. S. et al (1992), and Lambert, B. et al. (1992)). Advances in molecular biology recently overcame these limitations by making it possible to express proteins continuously in plants, which are then protected from specific insect pests (Estruch, J. J. et al. (1997)). The dramatic increase in worldwide utilization of Bt in agriculture following this innovation is testimony to the improvement it represents, and it signifies only the first step in utilizing protein engineering to realize the full potential of these environmentally benign pesticides. Higher molar activities, activities against a wider range of targets, increased stability, more efficient expression, and especially activities against new targets or targets which have developed resistance to other Bt toxins are all goals of programs to genetically engineer these proteins (Schnepf, H. E.(1995) and Thompson, M. A. et al. (1995)). Ideally, engineering of Bt toxins for improved performance would proceed by making targeted structural changes based on knowledge of structure-function relationships within the proteins and their mechanism of action. However, although detailed structural information is available for two Cry proteins to date, a profusion of mutagenesis studies aimed at revealing structure-function relationships in these proteins have produced confusing, sometimes conflicting results. The detailed structural information available is derived from the X-ray crystallographic analyses of the activated forms of Bt toxins Cry3A (Li, J. et al.) and Cry1Aa (Grochulski, P. et al. (1995). These studies revealed that the activated form (amino acid residues 33-609 of the Cry1Aa protoxin) of both of these polypeptides consists of three globular domains. This tertiary structure, as well as amino acid homologies and secondary structures within the domains led to assignment of putative functions for each. Originally, domain I was assigned the pore-forming role, domain II contained the hypervariable region and was designated as the determinant of receptor specificity, and domain III was thought to play a primarily structural role (Li, J. et al.(1991). The results of multiple mutagenesis studies and domain swapping experiments have now blurred these lines, especially for domains II and III (See Dean, D. H. et al. (1996) and Thompson, M. A. et al. (1995), for reviews). Mutations in the hypervariable region (designated loop 2) of domain II do indeed reduce receptor binding and toxicity (Rajamohan, F. et al.(1996) and Smith, G. P. et al. (1988)), and in a study of cross resistance to multiple Cry1 toxins, domain II was the essential determinant of toxicity (Tabashnik, B. E. et al.(1996)). However, in domain-swapping experiments coupled with in vitro binding studies, domain III correlated with receptor specificity (Bosch, D., et al. (1994); de Maagd, R. A., et al. (1996); and Lee, M. K. et al. (1995)), and mutations in domain III also reduced pore formation (Chen, X. J., et al.(1993) and Wolfersberger, M. G. et al. (1987)). Taken together, these results indicate an interdependence of the three domains and recommend that screening strategies for finding toxins with new properties must test whole Bt toxins (e.g., Bosch, D. et al. (1994)) rather than isolated domains in order to effectively assess a new toxin's potential. Thus, until the structure-function relationships within Bt toxins are better understood, improvement of these proteins remains largely dependent upon screening of large numbers of toxin variants for the properties required.

Several screening assays for Bt toxin effectiveness are presently available. The rate limiting step for all such assays, however, is the currently time-consuming task of preparing hundreds or thousands of samples of different activated toxins for testing. Purification of toxins from Bt is time consuming and requires different conditions for each toxin. Traditional E. Coli expression systems are faster by comparison, but only allow expression of protoxins which then must be solubilized, again often with individual conditions. Accordingly, there exists a continuing need for more efficient methods and materials to screen Bt toxins.

### BRIEF SUMMARY OF THE INVENTION

The subject invention concerns methods and materials for the simple expression and display of active protein toxins. One aspect of the subject invention pertains to a polynucleotide molecule that comprises a nucleotide sequence encoding an active Bt toxin coupled to a nucleotide sequence encoding a phage vector protein.

Another aspect of the subject invention pertains to cells transformed with a polynucleotide molecule as described above.

A further aspect of the subject invention pertains to a method of preparing active Bt toxins comprising transforming one or more cells with a polynucleotide molecule that comprises a nucleotide sequence which codes for an active Bt toxin and a nucleotide sequence which codes for a phage vector protein; and growing said one or more cells under conditions where said polynucleotide molecule is expressed, thereby forming a fusion protein having toxic activity or ability to bind to a toxin-specific target.

Further still, a different aspect pertains to a protein having a phage capsid region and a region capable of exhibiting pest control activity. The terms “pest control activity” and “toxin” or “toxic” activity are used interchangeably herein, and refer to the ability of a given protein to have an observable effect on pests.

Yet another aspect of the subject invention pertains to a method of screening for novel Bt toxins comprising contacting a polypeptide produced by the methods described herein with a target molecule and selecting those polypeptides capable of producing an observable effect. Preferably, the polypeptides of the subject invention are grouped in an expression library. Even more preferred, the polypeptides are compressed within a phage-display library.

A still further aspect of the subject invention pertains to a kit comprising a container, the container having disposed therein materials for producing or screening novel pest agents in accord with the teachings herein.

Moreover, another aspect of the subject invention pertains to one or more cells transformed with a polynucleotide molecule encoding a novel toxin obtained by the methods taught herein.

The subject invention provides a simple and efficient method of producing toxins that are soluble and ready to assay. This is a significant improvement over conventional expression systems which produce protoxins in crystals or inclusion bodies, which then must be solubilized before use. Further, because the purification protocols taught herein act on the phage particle rather than on the particular toxin which makes up only a very small portion of it, individual requirements for solubility and activation are not needed and therefore thousands of samples can theoretically be processed in parallel. Other advantages include the rapid and inexpensive purification procedure, the ability to quantitate toxin by phage titrating, the use of preparing single-stranded DNA for sequencing, and the assurance that most frame shift mutations will be eliminated automatically from toxin libraries. These and other advantages are further described below.

### BRIEF DESCRIPTION OF THE DRAWINGS

FIG. 1 depicts the amino acid sequences of Cry1Ac-pIII fusion protein as derived from the DNA sequences, as taught herein. W indicates a tryptophan substituted in place of a stop codon by the amber suppressor present in the host JM109 cells. Underlining indicates protein sequence which is present in neither the native Cry1Ac nor the cpIII protein. Indicates the predicted signal sequence protease cleavage site.

FIG. 2 shows the immunoblot detection of phage-displayed fusion proteins as taught herein. Phages (10⁹ TU for fUSE5 constructs and 10¹⁰ TU for SurfZAP constructs) were boiled from 4 min. In 30 μl of Laemmli denaturing sample buffer and size separated by SDS-8% PAGE. The proteins were transferred to nitrocellulose and detected by a rabbit polyclonal anti-Cry1Ac antibody and then by an alkaline phosphatase-conjugated goat anti-rabbit antibody. Lane 1, 1Ac-fUSE5 phage; lane 2, WT-fUSE5 phage; lane 3, 10 ng of purified trypsin-activated HD-73 Cry1Ac; lane 4, SurfZAP-FAb phage; lane 5, SurfZAP-1Ac phage; lane 6, 10 ng of purified trypsin-activated HD-73 Cry1Ac. The blot was divided through the molecular mass markers between lanes 3 and 4, and the two sides were developed separately. The SurfZAP side of the blot was allowed to develop for twice as long as the fUSE5 side, since SurfZAP-1Ac phage produced a lower-intensity signal. The effect of the longer development time can be observed by comparing the 10-ng toxin bands on each side of the blot (lanes 3 and 6).

FIG. 3 shows the ELISA detection of phage-displayed fusion proteins taught herein. Cry1Ac-expressing phage, 1Ac-fUSE5 and SZ-1Ac, and their respective control phages, WT-fUSE5 (no insert) and SZ-FAb (antibody insert), were compared with purified, trypsin-activated HD-73 Cry1Ac in an ELISA. The numbers on the horizontal axis refer to different units per well for the fUSES phage, SurfZAP phage, and purified Cry1Ac toxin as indicated on the figure. Ten times more SurfZAP (SZ) phage than fUSE5 phage was applied per well per dilution in order to obtain absorbance readings in the same range. For SurfZAP phage, the results from one trial are shown (n=2). For fUSE5 phage and HD-73 Cry1Ac, the means of results from two different trials are plotted (n=4). Mean A₄₅₀ (n=2) values for the individual trials for purified Cry1Ac were (trial 1) 0.021, 0.112, 0.366, 1.137, and 2.422 and (trial 2) 0.029, 0.109, 0.333, 1.170, and 3.426, for 0.1, 0.3, 1.1, 3.3, and 11.0 ng of toxin, respectively. Mean A₄₅₀ (n=2) values for the individual trials for 1Ac-fUSE5 phage were (trial 1) 0.173, 0.506, 1.243, and 1.521 and (trail 2) 0.213, 0.308, 1.04, and 2.255 for 3.3×10⁷, 1.1×10⁸, 3.3×10⁸, and 1×10⁹ TU, respectively. All A₄₅₀ values for WT-fUSE5 phage in both trials were below 0.050.

FIG. 4 shows the immunoblot detection of Cry1Ac and Cry1Ac phage binding to BBMV in the presence and absence of proteinase inhibitors. Cry1Ac-fUSE5 phage (3×10⁹ TU) or purified and trypsin-activated HD-73 Cry1Ac toxin (50 ng) was incubated with M. Sexta BBMV (100 μg of total protein). Experiments shown in panels A and B are identical except that in panel B proteinase inhibitors (500 μM phenylmethylsulfonyl fluoride and 5 mM benzamidine) were added to the BBMV prior to addition of toxin or phage. Lanes 1, prestained standard molecular massmarkers (stds; sizes are indicated on the left); lanes 2, HD-73 Cry1Ac toxin (50 ng); lanes 3, HD-73 Cry1Ac toxin pulus BBMV supernatant; lanes 4, HD-73 Cry1Ac toxin plus BBMV pellet; lanes 5, 1Ac-fUSE5 phage; lanes 6, 1Ac-fUSE5 phage plus BBMV supernatant; lanes 7, Ac-fUSE5 phage plus BBMV pellet; lanes 8, BBMV alone (100 μg of total protein), Phg. Phage; S. Supernatant; P. pellet.

### BRIEF DESCRIPTION OF THE SEQUENCES

SEQ ID NO. 1 is a nucleotide sequence of LK01 primer.

SEQ ID NO. 2 is a nucleotide sequence of LK02 primer.

SEQ ID NO. 3 is a nucleotide sequence of LK04 primer.

SEQ ID NO. 4 is a nucleotide sequence of LK03 primer.

SEQ ID NO. 5 is a nucleotide sequence of PPELB primer.

SEQ ID NO. 6 is a nucleotide sequence of PCRY1 primer.

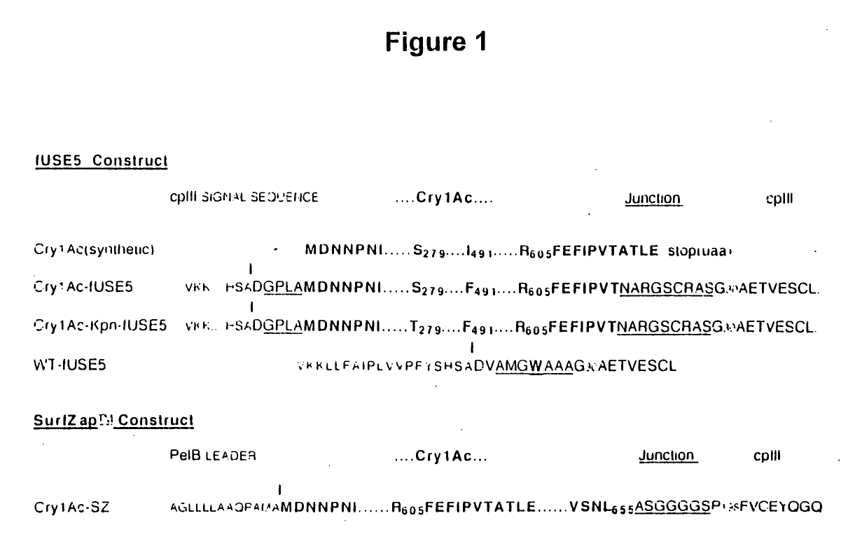

SEQ ID NO. 7 is an amino acid sequence of C-terminus of synthetic Cry1Ac from FIG. 1.

SEQ ID NO. 8 is an amino acid sequence of N-terminus of synthetic Cry1Ac from FIG. 1.

SEQ ID NO. 9 is an amino acid sequence of C-terminus of Cry1Ac-fUSE5 and Cry1Ac-Kpn-fUSE5 from FIG. 1.

SEQ ID NO. 10 is an amino acid sequence of N-terminus of Cry1Ac-fUSE5 and Cry1Ac-Kpn-fUSE5 from FIG. 1.

SEQ ID NO. 11 is an amino acid sequence of WT-fUSE5 from FIG. 1.

SEQ ID NO: 12 Is an amino acid sequence of C-terminus of Cry1Ac-SZ from FIG. 1.

SEQ ID NO: 13 is an amino acid sequence from internal portion of Cry1Ac-SZ from FIG. 1.

SEQ ID NO: 14 is an amino acid sequence of N-terminus of Cry1Ac-SZ from FIG. 1.

### DETAILED DISCLOSURE OF THE INVENTION

The subject invention is directed to methods and materials for the simple expression and display of active protein toxins, as exemplified by Bt toxins. One embodiment of the subject invention is directed to a novel polynucleotide molecule that comprises a nucleotide sequence encoding a Bt toxin and a nucleotide sequence encoding a phage vector protein. As described in the background of the invention, many Bt toxins have been isolated and sequenced. Polynucleotides encoding any known Bt toxins or those yet to be discovered and active fragments thereof (see, for example, U.S. Pat. No. 5,710,020) can be used in accord with the teachings herein. These include, but are not limited to, polynucleotides encoding Cry1Aa, CrylAb, Cry1Ac, Cry1B, Cry1C, Cry1E, and Cry3A. See Crickmore et al. (1998) for a description of other Bt toxins.

In another embodiment, the subject invention is directed to cells transformed with a polynucleotide encoding both a Bt toxin and a phage vector protein such that said polynucleotide molecule is expressed to make a fusion protein that exhibits pest control activity.

Another embodiment is drawn to one or more cells transformed with a polynucleotide encoding a novel toxin obtained by the methods taught herein. In a preferred embodiment, these cells are plant cells.

In another embodiment, the subject invention is directed to a method of preparing active Bt toxins comprising the steps of transforming one or more cells with a polynucleotide molecule comprising a nucleotide sequence encoding an active Bt toxin and a nucleotide sequence encoding a phage vector; and growing said one or more cells under conditions where said polynucleotide molecule is expressed thereby forming a fusion protein having toxic activity.

In a further embodiment, the subject invention is directed to a phage display library comprising a plurality of recombinant phage having a toxin displayed on the surface thereof. In a further embodiment, the subject invention is directed to a phage clone comprising a polynucleotide encoding a fusion protein having a toxin region.

Yet another embodiment is drawn to a method of screening novel toxins comprising obtaining a phage display library comprising a plurality of recombinant phage having a toxin displayed on the surface thereof, and screening said library to identify a phage clone comprising a phage vector which binds to a toxin-specific target.

A “phage library” is a protein expression library, constructed in a phage vector that expresses a collection of cloned protein sequences as fusions with a phage coat protein. Thus, in the context of the invention, proteins having ligand-binding potential are expressed as fusion proteins on the exterior of the phage particle. This disposition advantageously allows contact and binding between the recombinant binding protein and an immobilized ligand. Those having ordinary skill in the art will recognize that phage clones expressing binding proteins specific for the ligand can be substantially enriched by serial rounds of phage binding to the immobilized ligand, dissociation from the immobilized ligand, and amplification by growth in bacterial hose cells.

As used herein a “toxin-specific target” “target,” or “ligand” is a molecule or compound that can bind a phage vector recombinant protein produced according to the subject method.

Those having ordinary skill in the art will appreciate that the targets or ligands bound by the recombinant binding proteins of the present invention can be carbohydrates, lipids, or proteins. Further, these ligands can be either extracellular or intracellular molecules. Extracellular constituents are represented by molecular species attached to the exterior surface of the cell membrane. For Bt toxins this includes molecules in the apical microvillar membranes of the alimentary tract. Specific targets can include known Cry toxin binding molecules (aminopeptidases, cadherin-like proteins and lipids). Target ligands may be derived from preparations of brush border membrane vesicles. Intracellular targets can include molecules attached to the surface of organelle membranes.

Recombinant Vectors

Several types of vectors are available and may be used to practice this invention, including plasmid vectors and viral vectors. Plasmid vectors are the preferred vectors for use herein, as they may be constructed with relative ease, and can be readily amplified. Plasmid vectors generally contain a variety of components including promoters, signal sequences, phenotypic selection genes, origin of replication sites, and other necessary components as are known to those of ordinary skill in the art.

Vectors used herein are preferablyM13 phage vectors. Specific vectors contemplated for use herein include, but are not limited to, fUSE5, fAFF1, fd-CAT1, m663, 33, 88, Phagemid, pHEN1, pComb3, pComb8, pCANTAB 5E, p8V5, and ASurfZap.

Promoters most commonly used in prokaryotic vectors include the lac Z promoter system, the alkaline phosphatase pho A promoter, the bacteriophage .lambda.PL promoter (a temperature sensitive promoter), the tac promoter (a hybrid trp-lac promoter that is regulated by the lac repressor), the trypotophan promoter, and the bacteriophage T7 promoter. For general descriptions of promoters, see section 17 of Sambrook et al. (1989). While these are the most commonly used promoters, other suitable microbial promoters may be used as well.

One other useful component of vectors used to practice this invention is a signal sequence. This sequence is typically located immediately 5′ to the gene encoding the fusion protein, and will thus be transcribed at the amino terminus of the fusion protein. However, in certain cases, the signal sequence has been demonstrated to be located at positions other than 5′ to the gene encoding the protein to be secreted. This sequence targets the protein to which it is attached across the inner membrane of the bacterial cell. The DNA encoding the signal sequence may be obtained as a restriction endonuclease fragment from any gene encoding a protein that has a signal sequence. Suitable prokaryotic signal sequences may be obtained from genes encoding, for example, LamB or OmpF (Wong et al., (1997). Preferably the signal sequence is a cpIII signal sequence.

Another useful component of the vectors used to practice this invention is phenotypic selection genes. Typical phenotypic selection genes are those encoding proteins that confer antibiotic resistance upon the host cell. By way of illustration, the ampicillin resistance gene (amp), and the tetracycline resistance gene (tet) are readily employed for this purpose.

Construction of the polynucleotides contemplated herein can be prepared using standard recombinant DNA techniques as described in Sambrook et al., supra. Isolated DNA fragments to be combined to form the vector are cleaved, tailored, and ligated together in a specific order and orientation to generate the desired vector.

The DNA is cleaved using the appropriate restriction enzyme or enzymes in a suitable buffer. In general, about 0.2-1 mg of plasmid or DNA fragments are used with about 1-2 units of the appropriate restriction enzyme in about 20 ml of buffer solution. Appropriate buffers, DNA concentrations, and incubation times and temperatures are specified by the manufacturers of the restriction enzymes and are well-known by those skilled in the art. Generally, incubation times of about one or two hours at 37° C. are adequate, although several enzymes require higher temperatures. After incubation, the enzymes and other contaminants are removed by extraction of the digestion solution with a mixture of phenol and chloroform, and the DNA is recovered from the aqueous fraction by precipitation with ethanol.

To ligate the DNA fragments together to form a functional vector, the ends of the DNA fragments must be compatible with each other. In some cases, the ends will be directly compatible after endonuclease digestion. However, it may be necessary to first convert the sticky ends commonly produced by endonuclease digestion to blunt ends to make them compatible for ligation. To blunt the ends, the DNA is preferably treated in a suitable buffer for at least 15 minutes at 15° C. with 10 units of the Klenow fragment of DNA polymerase I (Klenow) in the presence of the four deoxynucleoside triphosphates. The DNA is then purified by phenol-chloroform extraction and ethanol precipitation.

The cleaved DNA fragments may be size-separated and selected using DNA gel electrophoresis. The DNA may be electrophoresed through either an agarose or a polyacrylamide matrix. The selection of the matrix will depend on the size of the DNA fragments to be separated. After electrophoresis, the DNA is extracted from the matrix by electroelution, or, if low-melting agarose has been used as the matrix, by melting the agarose and extracting the DNA from it, as described in sections 6.30-6.33 of Sambrook et al., supra.

The DNA fragments that are to be ligated together (previously digested with the appropriate restriction enzymes such that the ends of each fragment to be ligated are compatible) are put in solution approximately equimolar amounts. The solution will also contain ATP, ligase buffer, and a ligase such as T4 DNA ligase at about 10 units per 0.5 mg of DNA. If the DNA fragment is to be ligated into a vector, the vector is at first linearized by cutting with the appropriate restriction endonuclease(s). The linearized vector is then treated with alkaline phosphatase or calf intestinal phosphatase. The phosphatasing prevents self-ligation of the vector during the ligation step.

After ligation, the vector with the foreign gene now inserted is transformed into a suitable host cell. Prokaryotes are the preferred host cells for this invention. Suitable prokaryotic host cells include E coli strain JM109, E coli strain JM101, E. coli K12 strain 294 (ATCC number 31,466), E. coli strain W3110 (ATCC number 27,325), E. coli X1776 (ATCC number 31,537), E. coli XL-lBlue (Stratagene), and E. coli B; however, many other strains of E. coli, such as HB101, NM522, NM538, NM539, and many other species and genera of prokaryotes may be used as well. In addition to the E. coli strains listed above, bacilli such as Bacillus subtilis, other enterobacteriaceae such as Salmonella trphimurium or Serratia marcesans, and various Pseudomonas species may all be used as hosts.

Transformation of prokaryotic cells is readily accomplished using the calcium chloride method as described in section 1.82 of Sambrook et al., supra. Alternatively, electroporation (Neumann et al., (1982)) may be used to transform these cells. The transformed cells are selected by growth on an antibiotic, commonly tetracycline (tet) or ampicillin (amp), to which they are rendered resistant due to the presence of tet and/or amp resistance genes on the vector.

After selection of the transformed cells, these cells are grown in culture and the plasmid DNA (or other vector with the foreign gene inserted) is then isolated. Plasmid DNA can be isolated using methods known in the art. Two suitable methods are the small scale preparation of DNA and the large-scale preparation of DNA as described in sections 1.25-1.33 of Sambrook et al., supra. The isolated DNA can be purified by methods known in the art such as that described in section 1.40 of Sambrook et al., supra. This purified plasmid DNA is then analyzed by restriction mapping and/or DNA sequencing. DNA sequencing is generally performed by either the method of Messing et al., (1981) or by the method of Maxam et al., (1980) Meth. Enzymol., 65:499.

Gene Fusion

This invention contemplates fusing a polynucleotide encoding the polypeptide of interest (toxin) to a second polynucleotide encoding a phage protein such that a fusion protein is generated during transcription. The phage protein is typically a coat protein gene, and preferably it is the filamentous (fl) phage cpIII gene or a fragment thereof. Fusion of the toxin polynucleotide and the phage polynucleotide may be accomplished by inserting the phage polynucleotide into a particular site on a plasmid that also contains the toxin polynucleotide gene, or by inserting the toxin polynucleotide into a particular site on a plasmid that also contains the phage polynucleotide.

Insertion of a polynucleotide into a plasmid requires that the plasmid be cut at the precise location that the polynucleotide is to be inserted. Thus, there must be a restriction endonuclease site at this location (preferably a unique site such that the plasmid will only be cut at a single location during restriction endonuclease digestion). The plasmid is digested, phosphatased, and purified as described above. The polynucleotide is then inserted into this linearized plasmid by ligating the two DNAs together. Ligation can be accomplished if the ends of the plasmid are compatible with the ends of the polynucleotide to be inserted. If the restriction enzymes are used to cut the plasmid and isolate the gene to be inserted create blunt ends or compatible sticky ends, the DNAs can be ligated together directly using a ligase such as bacteriophage T4 DNA ligase and incubating he mixture at 16° for 1-4 hours in the presence of ATP and ligase buffer as described in section 1.68 of Sambrook et al., supra. If the ends are not compatible, they must first be made blunt by using the Klenow fragment of DNA polymerase 1 or bacteriophage T4 DNA polymerase, both of which require the four deoxyribonucleotide triphosphates to fill-in overhanging single-stranded ends of the digested DNA. Alternatively, the ends may be blunted using a nuclease such as nuclease S1 or mung-bean nuclease, both of which function by cutting back the overhanging single strands of DNA. The DNA is then religated using a ligase as described above. In some cases, it may not be possible to blunt the ends of the polynucleotide to be inserted, as the reading frame of the coding region will be altered. To overcome this problem, oligonucleotide linkers may be used. The linkers serve as a bridge to connect the plasmid to the polynucleotide to be inserted. These linkers can be made synthetically as double stranded or single stranded DNA using standard methods. The linkers have one end that is compatible with the ends of the polynucleotide to be inserted; the linkers are first ligated to this polynucleotide using ligation methods described above. The other end of the linkers is designed to be compatible with the plasmid for ligation. In designing the linkers, care must be taken to not destroy the reading frame of the polynucleotide to be inserted or the reading frame of the polynucleotide contained on the plasmid. In some cases, it may be necessary to design the linkers such that they code for part of an amino acid, or such that they code for one or more amino acids.

Between the toxin and phage polynucleotide, DNA encoding a termination codon may be inserted, such termination codons are UAG (amber), UAA (ocher) and UGA (opel). (Davis et al. (1980)). The termination codon expressed in a wild type host cell results in the synthesis of the toxin gene protein product without the phage polynucleotide protein attached. However, growth in a suppressor host cell results in the synthesis of detectable quantities of fused protein. Such suppressor host cells can contain a tRNA modified to insert an amino acid in the terminating codon position of the mRNA, thereby resulting in production of detectible amounts of the fusion protein. Such suppressor host cells are well known and described, such as E. coli suppressor strain (Bullock et al. (1987)). Any acceptable method may be used to place such a termination codon into the mRNA encoding the fusion polypeptide.

The suppressible codon may be inserted between the first gene encoding a toxin and a second polynucleotide encoding at least a functional portion of a phage coat protein. Alternatively, the suppressible termination codon may be inserted adjacent to the fusion site by replacing the last amino acid triplet in the toxin or the first amino acid in the phage coat protein. When the phagemid containing the suppressible codon is grown in a suppressor host cell, it results in the detectable production of a fusion polypeptide containing the toxin and the coat protein. When the phagemid is grown in a non-suppressor host cell, the polypeptide is synthesized substantially without fusion to the phage coat protein due to termination at the inserted suppressible triplet encoding UAG, UAA or UGA. In the non-suppressor cell, the polypeptide is synthesized and secreted from the host cell due to the absence of the fused phage coat protein which otherwise anchored it to the host cell.

Mutations of Toxins

A polynucleotide molecule encoding the toxin may be altered at one or more selected codons. An alteration is defined as a substitution, deletion, or insertion of one or more nucleotides in the gene encoding the toxin that results in a change in the amino acid sequence of the polypeptide. Preferably, the alterations will be by substitution of at least one amino acid with any other amino acid in one or more regions of the molecule. The alterations may be produced by a variety of methods known in the art. These methods include, but are not limited to, oligonucleotide-mediated mutagenesis, cassette mutagenesis, error-prone PCR, and DNA shuffling.

Oligonucleotide-Mediated Mutagenesis. Oligonucleotide-mediated mutagenesis is the preferred method for preparing substitution, deletion, and insertion variants of the toxin gene. This technique is well known in the art as described by Zoller et al., 1987. Briefly, the toxin polynucleotide is altered by hybridizing an oligonucleotide encoding the desired mutation to a DNA template, where the template is the single-stranded form of the plasmid containing the unaltered or native DNA sequence of the toxin gene. After hybridization, a DNA polymerase is used to synthesize an entire second complementary strand of the template will thus incorporate the oligonucleotide primer, and will code for the selected alteration of the toxin polynucleotide.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (1978).

To alter the native DNA sequence, the oligonucleotide is hybridized to the single stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the toxin polynucleotide and the other strand (the original template) encodes the native, unaltered sequence of the toxin polynucleotide. This heteroduplex molecule is then transformed into a suitable host cell, usually a prokaryote such as E. coli JM101 or JM109. After growing the cells, they are plated onto agarose plates and screened using the oligonucleotide primer radiolabelled with 32-Phosphate to identify the bacterial colonies that contain the mutated DNA.

The method described immediately above may be modified such that a homoduplex molecule is created wherein both strands of the plasmid contain the mutation(s). The modifications are as follows: The single-stranded oligonucleotide is annealed to the single-stranded template as described above. A mixture of three deoxyribonucleotides, deoxyriboadenosin (dATP), deoxyriboguanosine (dGTP), and deoxyribothymidine (dTTP), is combined with a modified thio-deoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101 or JM109, as described above.

Mutants with more than one amino acid to be substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

Cassette Mutagenesis. This method is also a preferred method for preparing substitution, deletion, and insertion variants of the toxin polynucleotide. The method is based on that described by Well et al. Gene, 34:315 1985. The starting material is the plasmid (or other vector) comprising the toxin polynucleotide desired to be mutated. The codon(s) in the toxin polynucleotide to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the toxin polynucleotide. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. This plasmid now contains the mutated DNA sequence of the toxin polynucleotide.

Error-prone PCR There are several protocols based on altering standard PCR conditions (Saiki et al. (1988)) to elevate the level of mutations during amplification. Usually, the rate of mutation during PCR is one nucleotide per 10 kb replicated (Keohavong and Thilly (1989)). However, when the concentration of deoxynucleoside triphosphate is increased and Mn²⁺ is added, the rate of mutation increases significantly to −7×10⁻³ per nucleotide (Cadwell and Joyce (1992)) for Taq DNA polymerase. Since the mutations are introduced at random without significant sequence bias, this is a convenient mechanism for generating populations of novel proteins (Cadwell and Joyce (1994)). On the other hand, error-prone PCR is not ideal for altering short peptide sequences because the number of mutations is low; this can be overcome somewhat by recursive rounds of error-prone PCR (Bartel and Szostak (1993)).

Not to be construed as limiting, the following steps represent a preferred example of performing Error-prone PCR:

1) Oligonucleotide primers are designed that flank the coding region of interest in the phage. They are preferably −21 nucleotides in length and flank the region to be mutagenized. The fragment to be amplified should also carry restriction sites within it to permit easy subcloning in the appropriate vector.

2) The following mixture is prepared:

30 pmol of each primer

20 finol of the DNA template

50 mMKCl

10 mMTris (pH 8.3)

7 mM MgCl₂

1 mMDTT

0.2 mM dATP, 0.2 mM dGTP, 1 mM dCTP, 1 mM TTP

Bring the final volume to 88 μl

3) 10 μl of 5 mM MnCl₂ is added and mixed in the mixture of step 2.

4) 5 units of Taq DNA polymerase is added.

5) The mixture of step 4 is cycled 24 times between 10 sec at 94° C., 30 sec 45° C., and 30 sec at 72° C. to amplify fragments up to 1 kb. For longer fragments, the 72° C. step can be lengthened by 30 sec for each kb.

6) The PCR product is digested with the appropriate restriction enzyme(s) to generate sticky ends. It may be advisable to gel purify the restriction fragment. Alternatively, use biotinylated oligonucleotide primers are used in step 1 for PCR; excess primers and the restriction enzyme generated terminal fragments can then be removed with streptavidin-agarose (Gibco: Cat. No. 15942-014). All of the primer molecules need to be biotinylated. Excess primers and terminal fragments can also be removed with Quick Spin columns (Boehringer-Mannheim) or a PCR cleanup kit (Qiagen).

7) The DNA segment is cloned into the appropriate vector by ligation.

8) The salts are removed from the ligating DNA, and the DNA segment is electroporated into bacterial cells.

DNA shuffling This method has been applied to interleukin 1β (IL-1β) (Stemmer (1994a)), and β-lactamase (Stemmer (1.994b)). In DNA shuffling, genes are broken into small, random fragments with DNase I, and then reassembled in a PCR-like reaction, but without any primers. The process of reassembling can be mutagenic in the absence of a proofreading polymerase, generating up to 0.7% error when 10- to 50-bp fragments are used. These mutations consist of both transitions and transversion, randomly distributed over the length of the reassembled segment.

Not to be construed as limiting, the following steps represent a preferred example of performing DNA shuffling:

1) The fragment to be shuffled is PCR amplified. Often it is convenient to PCR from a bacterial colony or plaque. Touch the colony or plaque with a sterile toothpick and swirl in a standard PCR reaction mix (10 mMTris HCl, 50 mMKCl, 1.5 mMMgCl₂, 0.2 mM each of DATP, dCTP, dGTP, and dTTP, 0.005% Brij 35, 0.1 to 1 μM of each primer). The reaction is heated for 10 min at 99° C. The reaction mix is cooled to 72° C., and 1-2 units of Taq DNA polymerase is added, followed by cycling the reaction 35 times for 30 sec at 94° C., 30 sec at 45° C., 30 sec at 72° C., and finally the sample is heated for 5 min at 72° C. (All the conditions here are for a 1-kb gene.)

2) The free primers are removed, preferably by purification of the DNA with Wizard PCR Preps (Promega, Madison, Wis.), or by gel purification. Complete primer removal is essential.

3) 2-4 μg of the DNA is fragmented with 0.15 units of DNase I (Sigma, St. Louis, Mo.) in 100 μl of 50 mM Tris HCl (pH 7.4), 1 mM MgCl₂, for 5-10 min at room temperature, freezed on dry ice, and thawed to continue digestion until desired size range is obtained. The desired size range depends on the application; for shuffling of a 1-kb gene, fragments of 100-300 bases are adequate.

4) The desired DNA fragment size range (100-300 bp) is gel purified from a 2% low-melting-point agarose gel or equivalent. The DNA pellet is then washed with 70% ethanol to remove traces of salt.

5) The DNA pellet is resuspended in PCR mix (Promega) containing 0.2 mM each dNTP, 2.2 mMMgCl₂, 50 mMKCl, 10 mMTris HCl, pH 9.0, 0.1% Triton X-100, at a high concentration of 10-30 ng of fragments per microliter of PCR mix (typically 100-600 ng per 10-20 μl PCR reaction). No primers are added in this PCR reaction. Taq DNA polymerase (Promega) alone can be used if a substantial rate of mutagenesis (up to 0.7% with 10- to 50-bp DNA fragments) is desired. The inclusion of a proofreading polymerase, such as a 1/30 (vol/vol) mixture of Taq and Pfu DNA polymerase (Stratagene, San Diego, Calif.) is expected to yield a lower error rate (Barnes (1994)) and allows the PCR of very long sequences. A program of 30-45 cycles of 30 sec 94° C., 30 sec 45-50° C., 30 sec 72° C. in an MJ Research PTC-150 minicucler (Cambridge, Mass.) is run. The progress of the assembly can be checked by gel analysis but this is normally not necessary. The PCR product at this point should contain the correct size product in a smear of larger and smaller sizes.

6) The correctly reassembled product of this first PCR is amplified in a second PCR reaction which contains the outside primers. Aliquots of 2.5 μl of the PCR assembly are diluted 40× with PCR mix containing 0.8 μM of each primer. A PCR program of 20 cycles of 30 sec 94° C., 30 sec 50° C., and 30-45 sec at 72° C. is run, with 5 min at 72° C. at the end. This amplification results in a large amount of PCR product of the correct size.

7) The best PCR product is then digested with terminal restriction enzymes, gel-purified, and cloned back into a phage or phagemid genome.

Following are examples which illustrate procedures for practicing the invention. These examples should not be construed as limiting, but rather as illustrating the broader teachings herein. All references cited herein are incorporated by reference to the extent they are not inconsistent with teachings herein.

### Materials and Methods

Constructs and Phage Preparation

fUSE5 system The fUSE5 filamentous phage vector and the methods for propagating both the vector and phage have been described (27, 34). We used a slight variation of this vector which, rather than a frameshifted spacer between the two SfiI cloning sites, contained an in-frame spacer fragment containing an amber codon (Howard Benjamin, unpublished). Host cells for all the experiments reported here were JM109 E. coli. Since JM109 cells contain an amber suppressor, the fuSE5 vector itself could produce viable phage, whereas in non-amber-suppressing host cells (eg. MC1061 E. coli) the amber codon between the two SfiI cloning sites in the pIII gene prevents translation of the pIII gene and therefore, production of viable phage. The phage produced by the fuSE5 vector in JM109 cells were used in these experiments as wild type (WT-fUSE5) phage. For construction of Cry1Ac-expressing phage, a synthetic gene for activated 65-kDa Cry1Ac toxin (patterned after the Bt subspecies kurstaki cry1Ac sequence, codon-optimized for high expression in plants (37), GenBank Accession number U63372), was amplified from plasmid pAGM19 with the primers LK01(5′-GTG AGT GAG TGG CCG ACG GGG CCG CTG GAA TGG ACA ACA ATCCCAACATC-3′) (SEQ ID NO: 1) and LK02 (5′-TGA GTG AGT CGG CCC CAG AGG CCC TGC AGC TCC CTC GAG CGT TGC AGT AAC GGG-3′)(SEQ ID NO: 2). These primers amplified the cry1Ac sequence (codons 1-616) and added SfI sites to each end (cry1Ac homologous sequence in the primers is underlined, SfI sites are italic). The 1.8-kb PCR product was digested with SfI and ligated to the 9.6-kb SfI digested fUSE5 vector, transformed into JM109 cells, and selected for growth on tetracycline media. Phage produced by fUSE5 do not kill their host cells and so grow as colonies on selective agar (35). Twenty colonies were selected at random and inoculated into 3 ml LB-tetracycline liquid cultures. The supernatants of these overnight cultures were screened for the presence of transducing units (tu) indicating production of functional phage. All of the supernatants were positive and contained approximately equal titers of phage. The cell pellets of the overnight cultures were processed for plasmid purification and the resulting DNA subjected to restriction analysis. The analysis revealed that 5 of the 20 colonies contained inserts of the appropriate 1.8-kb size, one had a slightly shorter insert, and the rest contained no insert. The five phage isolates carrying complete putative cry1Ac genes and a no insert fUSE5 control were purified from 50 ml cultures. Each was then individually combined with an artificial insect diet and fed to tobacco budworms (Heliothis virescens), a Cry1Ac-susceptible insect, in a simple single-dose feeding assay. The isolate found to be most toxic to the larvae (hereafter referred to as the 1Ac-fUSE5 phage) was sequenced through the cry1Ac and cpIII junction regions (Sequenase, USB) and kept for further experiments.

To create the 1Ac-Kpn-fUSE5 phage, which contains a unique KpnI site at the junction of domains I and II of Cry1 Ac, a G->C mutation was introduced into codon 279 by PCR mutagenesis as follows. The Cry1Ac gene in pAGM19 was amplified in two parts. Primers LKO1 (above) and LK04 (5′-GA GCC TCG AAA GGT ACC GTC-3′) (SEQ ID NO: 3) in one reaction amplified the 5′ end of the gene, codons 1 to 283. Primers LK02 (above) and LK03 (5′-GAC GGT ACC TTT CGA GGC TC-3′) (SEQ ID NO: 4) in a separate reaction amplified the 3′ end of cry1Ac, codons 277 to 613. The mismatching nucleotide in primers LK03 and LK04 is underlined. The whole gene was then reassembled in a third 100 ul PCR reaction containing 1 ul of each of the preceding two reaction products, and 10 pMoles each of primers LK01 and LK02. This 1.8-kb product was digested with SfiI and cloned into the SfiI sites of fUSE5. The entire sequence of the modified cry1Ac gene and the fusion junction with phage gene pIII was verified by DNA sequencing. This phage is referred to as 1Ac-Kpn-fljSE5 throughout this report.

Phage were purified by polyethylene glycol (PEG) precipitation (0.15 volumes of 16% [w/v] PEG 8000, 3.3 M NaCl) for 15 min on ice followed by centrifugation, and sometimes further re-precipitated with acetic acid (34).

SurfZap system The Stratagene Lambda SurfZAP™ vector is a 41.5-kb lambda phage vector derived from the LambdaZAP II™ vector (also Stratagene), which contains a defective filamentous phage (f1) genome that can be excised as a phagemid (pSurfscript) and packaged into f1 phage particles with the assistance of VCSM13 helper phage (17). A translational fusion of a cry1Ac gene with amino acids 198-406 of an f1 phage cpIII gene in the SurfZAP™ vector allows phage display of Cry1Ac protein on filamentous phage, and was constructed as follows. Codons 1 through 656 of a natural B. thuringiensis cry1Ac gene were PCR amplified from the OSU4202 construct (12) with primers that modified the ends as prescribed in the manufacturer's instructions. The upstream primer, PPELB (5′-CTCGCTCGCCCATAT/GCGGCCGC/AGGTC TCCTCCTCTTAGCAGCACAACCAGCAATGGCC/ATGGATAACAATCCGAACAT CAATGAATGC-3′) (SEQ ID NO: 5), provides a NotI site for ligation to the left lambda arm of SurfZAP, the remaining sequence to complete the pelB leader peptide (13 amino acids) and 30 nucleotides of homology to amino acids 1-10 of the cry1Ac coding region in frame with the pelB leader region (each segment delineated by “/”). The downstream primer, PCRY1(5′-ATCCGATAAATA/GCTAG C/TAAATTGGACACTTGATCAATATGATAATCCG-3′) (SEQ ID NO: 6), added an NheI site downstream of the XhoI site in cry1Ac which defines the C-terminal boundary of domain III of the active toxin and the protoxin coding region involved in crystal formation. The NheI site is just downstream of codon 656 of cry1Ac which, when ligated to the SpeI digested right lambda arm of SurfZAP, creates an in-frame fusion with the cpIII gene. NheI was chosen for the downstream primer as it creates a compatible cohesive end with SpeI of the vector and because the internal coding sequence of cry1Ac contains a SpeI site. The cry1Ac PCR product was digested with NotI and NheI, gel purified, ligated into the NotI/SpeI digested vector provided, and transformed into SOLR cells for excision of the phagemid. All further manipulations were as described in the manufacturer's protocols, including the construction and successful testing of positive control phage.

Bt Toxin Purification.

Cry1Ac toxin (65 kDa) was prepared from Bt subsp. kurstaki (HD-73) as described by Garczynski et al. (10) except that the final Sephacryl-300 column was omitted.

Insect Feeding Experiments

Toxicity of phage to insects was determined by insect feeding experiments as follows. Molten multi-insect diet (Southland Products, Lake Village, Ark.) was aliquotted into individual wells, approximately 2.5 ml diet per well for a feeding surface area of 1.8 cm²′ and allowed to solidify. Purified Bt Cry1Ac or phage were diluted in Tris-buffered saline (50 mM Tris [pH 7.4], 150 mM NaCl), and 50 ul aliquots were applied evenly to the diet surface in each well and allowed to dry. Dose groups for Bt Cry1Ac were 100 ng, 33 ng, 11 ng, 3.7 ng, and 1.2 ng per well. Dose groups for Cry1Ac-expressing phage were 10⁹, 3.3×10⁸, 1.1×10⁸, 3.3×10⁷, 1.1×10⁷ and zero transducing units per well. Control (WT-fUSE5) phage were tested at a single dose of 10¹⁰ phage per well. Newly hatched Heliothis virescens larvae (USDA Cotton Insects Research Laboratory; Stoneville, Miss.) were placed one per well on the treated diet. There were 20 insects per dose group or control group. Mortality was scored after incubation at 26° C. for 7 days, and LC₅₀'s calculated by probit analysis (29) with POLO PC software.

Preparation of Brush Border Membrane Vesicles (BBMV)

Midguts of fifth instar Manduca sexta larvae (Carolina Biologicals, Burlington, N.C.) raised on artificial multi-insect diet (Southland Products, Lake Village, Ark.) were removed, dissected free of peritrophic membrane and contents, rinsed briefly in cold grinding buffer (50 mM sucrose, 2 mM Tris-HCl [pH 7.4], with or without 500 μM phenylmethylsulfonyl fluoride and 5 mM benzamidine) and frozen on dry ice. The method of Wolfersberger et al. (43, see also 31) as modified by English and Readdy (7) was used for the isolation of brush border membrane vesicles, except that proteinase inhibitors were usually omitted to avoid inactivating phage and to attempt to assess the fate of phage ingested by susceptible insects. Briefly, frozen midguts were thawed in approximately 9 volumes ice cold grinding buffer and ground using a Dounce homogenizer (20 strokes). To this crude homogenate, CaCl₂ was added to 10 mM, followed by stirring on ice for fifteen minutes. The calcium treated homogenate was cleared by two centrifugations at 4300×g for 10 min at 5° C., discarding the pellet both times. Finally, BBMV were pelleted by centrifugation at 27,000×g for 10 min, 5° C., and resuspended in a small volume of 0.32 M sucrose with a Dounce homogenizer. If proteinase inhibitors were being used, they were re-added to the supernatant after each centrifugation and to the final suspension in sucrose. This suspension was stored in small aliquots at −70° C. Protein concentrations were determined by Biorad protein assay, using a bovine serum albumin standard curve.

ELISA

Enzyme-linked immunosorbant assays were performed essentially as described by Scott and Smith (34) except that phage and purified HD73 Cry1Ac controls were allowed to dry to the plate prior to addition of primary antibody. This reduced background. Phage were plated in duplicate at three, 3-fold dilutions from 10⁹ phage per well. HD73 Cry1Ac was plated at six, 3-fold dilutions from 100 ng per well. The primary antibody was the Protein-A sepharose purified IgG fraction of polyclonal rabbit anti-Cry1Ac anti-sera R118 (K. Luo, University of Georgia-Athens, unpublished) diluted 1:1500. Goat anti-rabbit alkaline phosphatase conjugated secondary antibody was obtained from Sigma, and used at 1:4000 dilution. Plates were developed for 10 min with p-nitro-phenyl phosphate (Sigma) and absorbances read at 405 nm (14).

Immunoblot Analysis

Protein immunoblot analysis was carried out according to standard techniques (14). Phage particles, HD73 Cry1Ac, and BBMV (see figure legends for amounts) were boiled 3 min in denaturing sample buffer (60 mM Tris-Cl [pH6.8], 10% glycerol, 2% sodium dodecyl sulfate [SDS], 0.05% bromophenol blue, 2.5% β-mercaptoethanol) and size-separated by electrophoresis on 8% or 110%-polyacrylamide SDS gels as noted in figure legends. Proteins were electrophoretically transferred to supported nitrocellulose (MSI), and the membranes blocked with 0.2% Tween-20 in Tris-buffered saline. Complete transfer was monitored by the movement of prestained protein standards (Sigma) to the membrane. Antibodies and their dilutions were the same as those for ELISA assays (above). All antibody incubations and membrane washes were in TBS-0.2% Tween-20. Blots were developed with nitroblue tetrazolium chloride and 5-bromo-4-chloro-3-indoylphosphate-ρ-toluidine salt (Sigma) in alkaline phosphate substrate buffer (100 mM Tris-HCl pH 9.5, 100 mM NaCl, 50 mM MgCl₂) and photographed.

Micropanning

Micropanning (3, 27) with the Cry1Ac-expressing phage (both fUSE5 and SurfZAP types) was attempted under a variety of conditions against a variety of targets. In general, phage and target molecules were incubated and allowed to bind in Tris-buffered saline (50 mM Tris [pH 7.4], 150 mM NaCl). Wash steps were rapid (6-10 washes in 10 minutes or less), and elution was achieved with 100 mM glycine [pH2.2] for 10 minutes, then neutralized with 1 M Tris base. In the case of micropanning against BBMV, the vesicles could be pelleted easily in a microcentrifuge at 13,000 rpm for 30 sec. Washes were therefore carried out at room temperature by repeated spins to pellet the BBMV, removal of supernatant, and resuspension in wash buffer by vigorous pipetting. Bound phage were eluted from BBMV by resuspending the washed pellet in 50 ul glycine (100 mM, pH 2.2) for 10 min, then adding 50 ul of 0.1% n-octyl-β-D-glucopyranoside to solubilize the vesicles. A 1 min spin pelleted insoluble debris, and the 100 ul supernatant was moved to a fresh tube, neutralized with 1 M Tris base, and titered immediately. Controls demonstrated that n-octyl-β-D-glucopyranoside was not toxic to phage at the concentration used.

Binding Studies with Iodinated Phage

Polyethylene glycol purified 1Ac-fUSE5 and WT-fljSE5 phage were radiolabeled with ¹²⁵Iodine by the chloramine-T method (18). BBMV binding assays were performed as previously described (10).

### EXAMPLE 1

### Production of Fusion Proteins

Phage expressing 65-kDa Cry1Ac toxin protein were produced in two different vector systems, the filamentous phage fUSE5 vector of Scott and Smith (34) and the lambda phage SurfZAP™ phagemid system of Stratagene (17). In both vector systems, we constructed a translational fusion of a gene encoding the active 65 kDa core of Cry1Ac toxin with a sequence encoding aminor filamentous phage coat protein, also known as the attachment protein, cpIII (or pIII). Both systems also fused a signal sequence, for transport out of the cell membrane, to the N-terminus of the Cry1Ac protein. In the fUSE5 vector this signal sequence is the native signal peptide of cpIII. In the SurfZAP vector the signal sequence provided is from the protein pelB. In both cases, the signal peptide is cleaved from the fusion protein during maturation of the phage particle leaving the Cry1Ac portion of the protein exposed at the free N-terminus. The cpIII portion of the fusion protein, located at the C-terminus of Cry1Ac, is partially buried in the capsid of the phage.

Despite the similarities described above, we chose to develop both systems because they differ in important ways which confer particular advantages to each for the expression of the Cry1Ac protein. The advantage of the SurfZAP vector, which is available as a kit, is that it is a phagemid system making use of a helper phage to package a phagemid DNA. Since this results in one or fewer fusion proteins being incorporated into any one phage particle along with 3-4 copies of native cpIII, the fusion protein does not need to be functional as an attachment protein, the function of native cpIII, in order for the recombinant phage to be propagated. In addition, having the cpIII gene isolated from the rest of the phage genome on the phagemid might simplify mutagenesis steps to create libraries of Cry1Ac variants. However, a disadvantage of this system is that phagemids carrying mutations in the fusion protein gene which result in truncations or frame-shifted reading frames will not be detected as such by observation of phage titers, nor will they be easily eliminated from the phage pool.

The fUSE5 vector in contrast, encodes an entire filamentous (fd) phage genome and therefore does not require helper phage. Since there is no helper phage, all phage carrying a recombinant vector express a recombinant cpIII, and all copies of cpIII on recombinant phage are fusion proteins. This is an advantage for detection of the presence of fusion protein by ELISA and immunoblot, since ten to fifty times fewer phage are required to accumulate the same amount of fusion protein as in the SurfZAP system. Binding studies utilizing iodinated phage are easier in the absence of the large percentage of non-expressing phage produced by the SurfZAP system. And finally, clones expressing truncations or frameshifted reading frames are automatically eliminated from the phage pool since they will not produce infectious phage. However, there was evidence that in our hands that expression of the large fusion protein on all five phage attachment spikes was a disadvantage for growth. Colonies formed by 1Ac-fUSE5 versus WT-fUSE5 infected cells on LB-tetracycline agar were not distinguishable. However, WT-fljSE5 plated on XL1-Blue (Tetracycline-resistant) cells on LB-tetracycline agar form plaques, and under those conditions the plaques produced by 1Ac-fUSE5 phage were approximately one fourth the diameter of those produced by the wild type cpIII expressing phage (L. K., data not shown). This suggests that expression of Cry1Ac in this system could lead to selection for in-frame deletion mutants that eliminate some or all of the Cry1Ac insert. PCR verification of insert size did show that 20 out of 20 randomly tested Cry1Ac-expressing phage had maintained their full-size Cry1 Ac insert after one round of amplification, however, so the extent of this selection is less than 5% per generation (L. K., data not shown).

In anticipation of creating libraries of Cry1Ac variants by mutagenesis of the fuSE5-expressed gene, a modified cry1Ac gene was also inserted in the fuSE5 vector for testing. This cry1Ac gene had a unique KpnI site added at the approximate junction of domains I and II of the Cry1Ac protein, estimated to be near amino acid Ser₂₇₉ according to crystallography data for a similar crystal protein, Cry1Aa (13). Since domains II and III, but not domain I are thought to play the major role in insect specificity, it was reasoned that creation of a removable domain II-III cassette would simplify later library construction. Insect feeding assays with this phage (1Ac-Kpn-fuSE5) (Table I) demonstrated that the S279->T₂₇₉ change required to create the KpnI restriction site did not reduce the toxicity of the protein.

The entire cry1Ac sequence in the fuSE5 vector and the fusion junctions in both vector systems were verified by DNA sequencing. FIG. 1 shows the sequences for WT-fUSE5, 1Ac-fuSE5, 1Ac-Kpn-fuSE5 phage and the Cry1Ac-expressing phage from the SurfZAP vector (1Ac-SZ). The fuSE5 Cry1Ac fusion sequences contained five additional amino acids at the N-terminus due to the addition of the SfiI cloning site in the vector. In addition, a single nucleotide change from the reported cry1Ac sequence, resulting in the single amino acid change I₁₄₉->F₄₉₁, was detected in both the 1Ac-fuSE5 and the 1Ac-Kpn-fUSE5 phage. This change was apparently present in the source plasmid pAGM19, since these two phage constructs were made from cry1Ac fragments amplified in independent PCR reactions containing pAGM19. Finally, the fusion junction with cpIII in the fUSE5 constructs contained some interesting unplanned changes. Originally designed so that there would be a Gly-Ala-Gly-Ala spacer between the Cry1Ac and cpIII peptide sequences, a deletion inducing a frame shift occurred in the fifth to the last codon of the cry1Ac gene of both constructs, possibly by an error in the primer LK02. Selection for viable infective phage particles required that the shift be corrected by matching insertions or deletions downstream to have a functional cpIII section of the protein. The result was the 11 amino acid spacer underlined in the figure, which is neither Cry1Ac nor cpIII sequence, followed by cpeIII starting at amino acid 30. This particular corrective rearrangement was selected three times independently during construction of different recombinant phage, emphasizing the power of the selection for phage viability built into this vector. Possible reasons for selection of this particular rearrangement are discussed below. Phage titers for this construct in JM109 cells were between 10⁹ and 10¹⁰ tu per ml of overnight culture.

The cry1Ac and fusion junction sequences for the 1Ac-SZ construct were found to be as planned (Table 1), with the fusion protein containing 43 additional C-terminal amino acids of Cry1Ac (a protoxin fragment) not in the fUSE5 constructs, an alanine/glycine spacer, and only the last 209 amino acids (198-406) of cpIII. The PelB leader peptide is presumed to be cleaved exactly at the N-terminus of the Cry1Ac portion of the protein, such that the natural end of the protein is exposed, unlike in our fuSE5 construct. Phage titers of this construct were usually over 10¹¹ phage per ml of culture.

### EXAMPLE 2

### Cry1Ac Expressing Phage, but Not Wild Type Phage, are Toxic to Manduca Sexta and Heliothis virescens

DNA sequencing verified that both the fUSE5 and SurfZAP constructs contained in frame cry1Ac-cpIII gene fusions. The ability of the resulting fusion proteins to fold into biologically active conformations was shown by the ability of our Cry1Ac-expressing phage preparations, and not control phage preparations, to kill insects susceptible to native Bt Cry1Ac. The toxicities of Cry1Ac-expressing phage as compared to purified HD73 Cry1Ac protein were determined by insect feeding assays. Phage precipitated from E. coli supernatants were titered to determine their concentration, then diluted in Tris-buffered saline and applied to the surface of insect diet in doses ranging from 109 transducing units (tu) to 10⁷ tu per well, with approximately 1.8 square cm of feeding surface area per well. Twenty larvae (H. virescens or M. sexta) were fed individually for each dose and control group for fuSE5 phage, and mortality recorded after seven days. LC₅₀'s were determined by probit analysis of the mortality data and are presented for H. virescens in Table I. Given that there are on average five molecules of the Cry1Ac-cpIII fusion protein per phage particle, and that it has been shown that typical filamentous phage preparations contain 20 phage particles for every transducing unit (35), it can be calculated that 10⁹ tu of 1Ac-fUSE5 phage contain approximately 10.4 ng of Cry1Ac protein (17.1 ng of the fusion protein). Therefore, the LC₅₀ of the phage expressed protein can also be expressed as 7.3 ng/well, in remarkably close agreement with the LC₅₀ for the purified Bacillus protein (7.6 ng/well). Likewise, conversion of the 1Ac-Kpn-fUSE5 LC₅₀ from transducing units to nanograms gives a dose of 7.5 ng per well. If these doses are expressed in terms of surface area, they average 4.2 ng per square cm, in agreement with published toxicity for this Cry protein against H. virescens (10). Single dose feeding experiments demonstrated that 1 Ac-fUSE5 phage were extremely toxic to Manduca sexta larvae as well (not shown). LC₅₀'s were not determined for SurfZAP phage, but 1Ac-SZ was shown to be toxic to H. virescens at adequate dosages.

TABLE 1
Results of insect feeding experiment with tobacco bud worm
comparing insecticidal activities of Cry1Ac protein and
Cry1Ac-expressing phage

| | LC₅₀a | LC₅₀a |
|---|---|---|
| Protein or phage | (95% Ci^{b} | (pmol of Cry1Ac) |
| Cry1Ac protein | 7.6 (3.5 − 13.7) ng | 11.7 |
| 1Ac-fUSE5 phage | 7.0 (4.2 − 11.0) × 10⁸ TU | 11.2 |
| 1Ac-Kpn-fUSE5 phage | 10.7 (7.2 − 15.8) × 10⁸ TU | 17.1 |
| Wild-type phage | None detected^{c} | Not applicable |

aDetermined by the probit analysis (29). All doses are expressed on a per well basis and were surface applied (appropriatmate surface area, 1.8 cm² per well.
^{b}CI, confidence interval.
^{c}Forty insects fed 10¹⁰ TU of WT-fUSE5 each (14 times the LC₅₀ of 1Ac fUSE5) showed 0% mortality and 2.5% growth inhibition.

### EXAMPLE 3

### Fusion Proteins Are Expressed on the Phage Particles and are Recognized by Cry1 ac Specific Antibodies

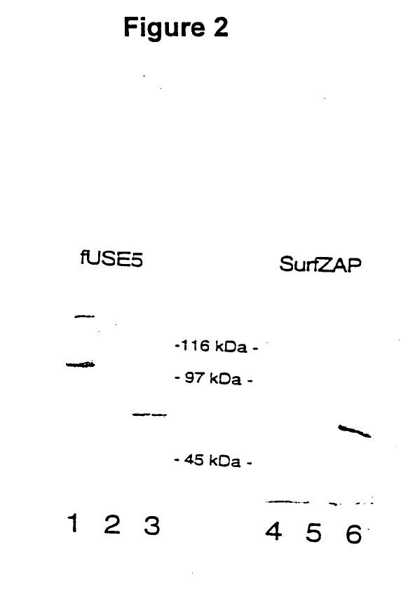

To confirm that the toxin was being expressed as a fusion protein incorporated into phage particles, purified phage were analyzed by immunoblotting. Phage particles produced in both the fUSE5 and SurfZAP systems were concentrated by precipitation in salt and acetic acid, pelleted, boiled in denaturing sample buffer, and subjected to SDS polyacrylamide electrophoresis. Proteins electrophoretically transferred to nitrocellulose were detected with rabbit anti-Cry1Ac antibodies (FIG. 2). HD73 Cry1Ac purified from Bt crystals was run in neighboring lanes for comparison of size and quantity (lanes 3 and 6). Fusion proteins were detected in the Cry1Ac-expressing phage of both vectors (lanes 1 and 5), but not in control phage (lanes 2 and 4). The mobility of the fUSE5 fusion protein indicated a molecular size of approximately 104-kDa, very close in size to the 107-kDa protein expected (65-kDa Cry1Ac plus 42-kDa cpIII). The difference may indicate some proteolysis of the phage, however it is also within the limits of accuracy for this type of determination and the sharpness of the band does not indicate proteolysis. A second, weaker band with an apparent molecular size of 130 kDa was also recognized by anti-Cry1Ac antibodies whenever immunoblotting was performed on 1Ac-fUSE5 phage. Its components were not positively identified, but the fact that it is twice the molecular weight of the 65 kDa toxin and is recognized by the anti-Cry1Ac anti-sera suggests that it may be an insoluble toxin dimer, resulting from proteolysis of fusion proteins by endogenous E. coli proteases. It has been previously observed in our lab that toxin associated with membranes can form aggregates that persist through SDS-cracking buffer treatment and appear as a >100-kDa band by SDS-PAGE (Y-J Lu, M. Adang, unpublished). Such aggregates are most likely inactive. Immunoblotting of 1Ac-Kpn-fUSE5 phage produced a banding pattern identical to 1Ac-fUSE5 phage (not shown). The SurfZAP fusion protein ran with an apparent molecular size of 117-kDa, somewhat larger than the expected molecular weight, since although this construct expresses 43 more amino acids of Cry1Ac than 1Ac-fUSE5 does, it includes only a 25 kDa fragment of cpIII. However, in neither the 1Ac-SZ nor the 1Ac-fUSE5 lanes was there any evidence that normal Cry1Ac protein not part of a fusion protein was being expressed. In addition, the relative quantities of Cry1Ac in the phage were as predicted in so far as the 1Ac-SZ fusion protein is expected to be present at 2% of the level of the fUSE5 fusion protein, and 10 times as many phage particles and twice as much development time was required for 1Ac-SZ to be detected.

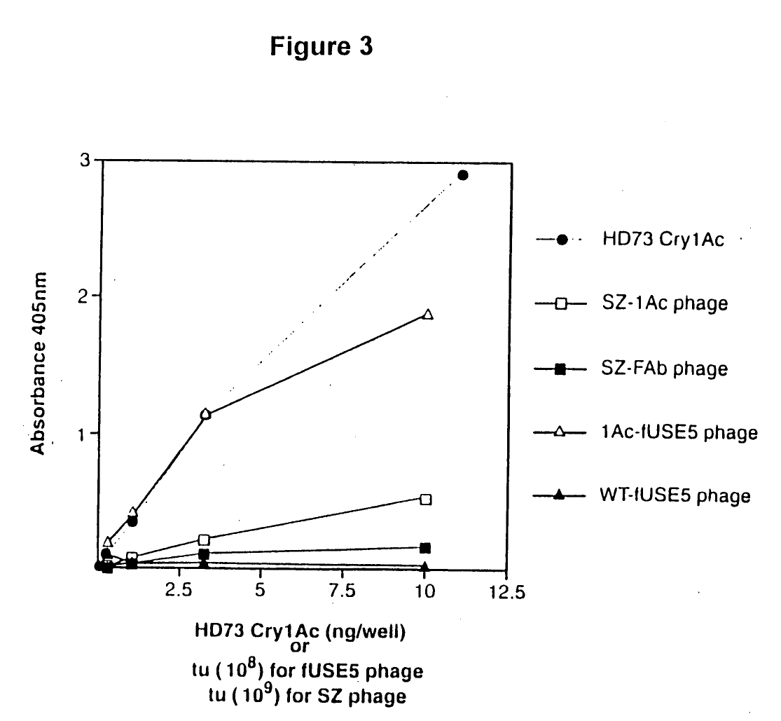

An ELISA was also performed on the Cry1Ac-expressing phages, since unlike the immunoblot, the proteins would not be denatured before antibody binding and quantitation could be more precise. Phage were serially diluted in Tris buffer and purified HD73 Cry1Ac protein served as a standard. Anti-Cry1Ac antibody was again used as the primary antibody. FIG. 3 is an average of two experiments for fuSE5 phage, both of which determined all points in duplicate. The SZ phage were included in one of these experiments. In this assay, both 1Ac-SZ and 1Ac-fuSE5 phage behaved exactly as expected for equivalent amounts of Cry1Ac protein not attached to phage, except at the highest phage concentrations at which the amount of other phage proteins in the well began to interfere with antibody binding. At those concentrations, however, the HD-73 Cry protein signal was also beyond the most linear part of its response curve. In the linear portion, the calculations arrived at above of the amount of Cry1Ac toxin expected to be present on a given amount of phage (eg. approximately 10 ng Cry1Ac per 10⁹ tu 1Ac-fuSE5 and 0.2 ng per 10⁹ tu 1Ac-SZ) were shown to be accurate. That is, observe in FIG. 3 that 3×10⁸ tu 1Ac-fUSE5 produced the same signal as 3 ng Cry1Ac toxin. Likewise, 3×10⁹ tu 1Ac-SZ produced a signal 20% as strong as that of 3×10⁸ tu 1Ac-fUSE5, or 2% when corrected for the number of phage. Therefore, since the amount of Cry1Ac present in our PEG purified phage preparations was in each case directly related to the phage titer, the Cry1Ac expressed from both the phagemid and phage vectors must be present in these preparations exclusively as fusion protein incorporated into phage particles.

In addition, both immunoassay results demonstrate that Bt toxin displayed on phage is suitable for these assays without any need to separate it from the phage particle, and therefore could go from overnight cultures into such assays with less than 30 minutes preparation time.

### EXAMPLE 4

### Micropanning Experiments

The possibility of affinity selection of Bt toxins by biopanning was tested under several conditions in micropanning experiments, since a library of phage displayed toxins had not been completed at this time. Micropanning is the comparison of the number of Cry1Ac-phage versus control phage bound by a toxin-specific target, such as anti-Cry1Ac antibody. It is a useful first step in biopanning since it helps establish the binding and elution conditions best suited to the affinity of the protein interaction under selection. Micropanning is also used to compare multiple candidate phage selected by a library biopanning, where addition of a competing ligand allows determination of the degree of specific binding of each phage (3).

Micropanning against an anti-Cry1Ac antibody was performed in duplicate in microtiter wells, 1 ug antibody per well, using bovine serum albumin as a blocking agent and control ligand. Results are summarized in Table II. The 1Ac-fjSE5 phage were preferentially retained by the antibody compared to control phage, and this binding was competed quite well (68%) with free Cry1Ac protein, indicating specific binding (Table II).

Micropanning with various preparations of toxin receptors proved more complex. When purified M sexta aminopeptidase-N, a known protein receptor for Cry1Ac in this species (19, 30), was micropanned in microtiter plates, no enrichment of 1 Ac-phage (fuSE5 or SurfZAP) over control phage was observed (data not shown). However, binding of the receptor protein directly to microtiter plates was probably not the ideal method to present this target for two reasons. First, it provides no control over the orientation of the protein on the plate. Second, binding of Cry1Ac toxin to soluble aminopeptidase-N has been shown to be only partly reversible (45%) (25), which would interfere with recovery of the affinity selected phage since our elution conditions would not be expected to release the receptor from the plates. Utilizing a biotin-streptavidin biopanning method (27) should overcome these problems.

Brush border membrane vesicles (BBMV) have been used as a source of toxin receptor in situ, and therefore were also tried as a biopanning target. In the absence of a suitable negative control, an excess of free Cry1Ac toxin was added to half the samples as a competitor. As with the purified receptor, there was no significant difference in the number of Cry1Ac-expressing phage versus control phage eluted after unbound phage were washed away (Table IIb). Two experiments reported below, a binding assay utilizing iodinated phage and an immunoblot of phage incubated with BBMV, uncovered possible reasons for this lack of affinity selectivity for BBMV.

TABLE 2
Micropanning with Cry1Ac-expressing phage
against anti-Cry1Ac antibody or BBMVl

| | Relative yield^{b} of: | |
|---|---|---|
| Target^{a} | WT-fUSE5 phage | 1Ac-fUSE5 phage |
| Bovine serum albumin | 1.0 | 1.0 |
| Anti-Cry1Ac Ab R118 | 0.5 | 7.1 |
| Anti-Cry1Ac Ab + 1 μg of Cry1Ac | 0.2 | 2.3 |
| 100 μg of BBMV | 1.0 | 1.0 |
| 100 μg of BBMV + μg of Cry1Ac | 0.24 | 1.7 |

^{a}Ab, antibody
^{b}Relative yields of control samples are set to 1.0.

### EXAMPLE 5

### Competition Binding of ¹²⁵i-radiolabeled Phage to Brush Border Membrane Vesicles

HD73 Cry1Ac purified from Bt, 1Ac-fUSE5 phage, and WT-fljSE5 control phage were iodinated by standard methods. Equal amounts of Manduca sexta BBMV, with or without unlabeled competitor HD73 Cry1Ac, were incubated with the iodinated phage or toxin. The results are presented in Table III. 1Ac-fUSE5 phage and HD73 Cry1Ac showed similar percentages bound, about 12%, which was approximately twice the percentage bound of the control phage, indicating some selective advantage of the Cry1Ac-expressing phage. However, whereas iodinated HD73 Cry1Ac was competed quite well by unlabeled toxin, the 1Ac-fUSE5 phage were not. One interpretation of this result is that non-specific binding interactions are occurring between the BBMV and the phage capsid, interfering with affinity selection under our binding conditions. It is possible of course, that other conditions could be found which could eliminate this problem.

### EXAMPLE 6

### Incubation of 1Ac-fuse5 Phage with Brush Border Membrane Vesicles Results in Proteolysis of the Cry1Ac-cpIII Fusion Protein

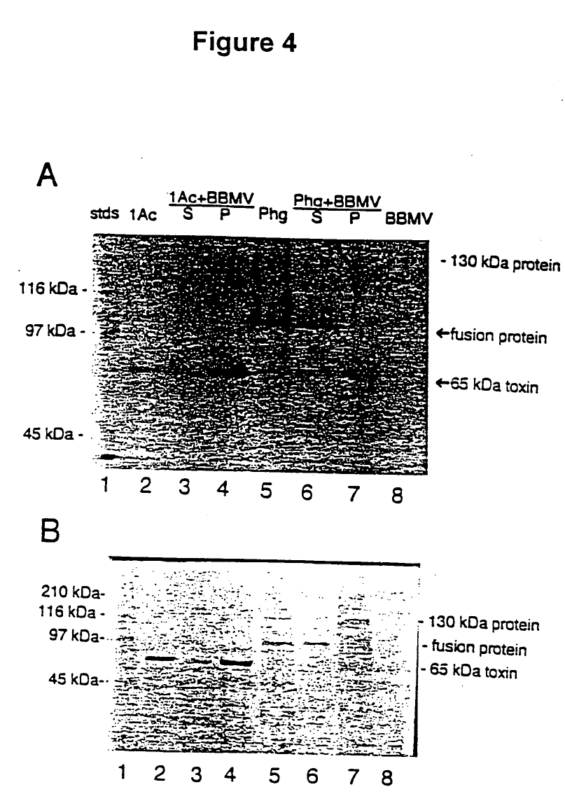

An essential step in the toxic mechanism of Bt crystal proteins, such as Cry1Ac, is proteolytic processing in the insect gut. Brush border membrane vesicles prepared from insect midgut in the absence of proteinase inhibitors (as they were in the experiments above) may more closely approximate the conditions the toxin encounters when ingested than proteinase-inhibited preparations. However, the proteinases associated with BBMV might interfere with biopanning by degrading phage capsid proteins or the fusion protein. To determine the fate of the Cry1Ac-cpIII fusion protein in the presence of BBMV, 1Ac-fUSE5 phage were incubated at room temperature with BBMV without proteinase inhibitors, as if micropanning, but then subjected to immunoblot analysis to detect Cry1Ac and fusion protein. Equivalent amounts of Cry1Ac toxin were analyzed in parallel. To separate toxin or phage bound to the BBMV from the unbound, the BBMV were pelleted and the supernatants analyzed separately from the vesicle pellets (which were also washed before analysis). The results were surprising (FIG. 4A). Purified toxin (panel A, lane 2, control) when incubated with BBMV, was found to segregate with the pellet (panel A, lane 4), apparently bound to the vesicles. In contrast, after incubation of 1Ac-fUSE5 phage (panel A, lane 5, control) with BBMV, 104 kDa fusion protein was detected only in the supernatant (panel A, lane 6). Also in the supernatant, were smaller amounts of a 65 kDa protein recognized by anti-Cry1Ac antibodies, which is most likely the Cry1Ac portion of the fusion protein released by proteolysis. The pellet contained only the 65 kDa protein (panel A, lane 7). No proteins were recognized by anti-Cry1Ac antibodies in the BBMV control (panel A, lane 8). The presence of some free toxin in the supernatant (panel A, lane 6) and absence of fusion protein in the pellet (panel A, lane 7) suggests that proteolysis of the fusion protein occurs before binding of the toxin portion to BBMV. To test this hypothesis, purified toxin and 1 Ac-fUSE5 binding experiments were repeated in the presence of proteinase inhibitors (FIG. 4., panel B). Proteinase inhibitors did not alter purified toxin binding to BBMV (panel B, lane 4). However in contrast to results seen without inhibitors, no 65 kDa protein was detected in the phage samples by anti-Cry1Ac antibodies in either the supernatant (panel B, lane 6) or the BBMV pellet (panel B, lane 7). The 104 kDa fusion protein was clearly detected in the supernatant, but no clear fusion protein band was seen in the pellet. The faint 130 kDa band was detected in both the pellet and supernatant fractions. With regard to biopanning, these results indicate that BBMV failed as a source for target ligands because it did not bind whole phage.

TABLE 3
Competition binding of ¹²⁵I-radiolabelled phage to BBMV

| Radiolabelled | 100-fold | | | | |
|---|---|---|---|---|---|
| toxin or phage | excess | | Mean cpm | | % |
| added to 10 μg | of unlabeled | cpm | bound | % cpm | Compe- |
| of BBMV | Cry1Ac | added | (n = 2) | bound | tition |
| ¹²⁵I-Cry1Ac | − | 98,242 | 12,690 | 12.9 | 66.5 |
| protein | + | 98,242 | 4,256 | 4.3 | |
| ¹²⁵I-1Ac-fUSE5 | − | 105,252 | 12,293 | 11.7 | 8.5 |
| phage | + | 105,252 | 11,212 | 10.7 | |
| ¹²⁵I-WT-fUSE5 | − | 62,319 | 3,784 | 6.1 | None |
| phage | + | 62,319 | 4,743 | 7.6 | |

It should be understood that the examples and embodiments described herein are for illustrative purposes only and that various modifications or changes in light thereof will be suggested to persons skilled in the art and are to be included within the spirit and purview of this application and the scope of the appended claims.

### REFERENCES

Adang, M. J. (1991) “Bacillus thuringiensis insecticidal crystal proteins gene structure, action, and utilization,” Biotechnology for Biological Control of Pests and Vectors, K. Maramorosch ed. CRC Press, Boca Raton. pp. 3-24.

Bartel, D., and Szostak, J. (1993) “Isolation of new ribozymes from a large pool of random sequences,” Science 261:1411-1418.

Benjamin, Howard, Praecis Pharmaceuticals, Inc., Cambridge, Mass.; unpublished)

Bosch, D., B. Schipper, H. Van Der Kleij, R. A. De Maagd, and W. J. Steikema (1994) “Recombinant Bacillus thuringiensis crystal proteins with new properties Possibilities for resistance management,” Biotechnology 12:915-918.

Bullock et al., Bio Techniques 5, 376-379 1987

Cabilly, S., (Eds) (1997) “Methods in Molecular Biology Combinatorial Peptide Library Protocols,” Humana Press, Clifton, N.J. p. 320.

Cadwell, R., and Joyce, G. F. (1992) “Randomization of genes by PCR mutagenesis,” PCR Methods Appl. 2:28-33.

Cadwell, R., and Joyce, G. (1994) “Mutagenic PCR,” PCR Methods/Appl. 32:S136-S140.

Chen, X. J., M. K. Lee, and D. H. Dean (1993) “Site-directed mutations in a highly conserved region of Bacillus thuringiensis d-endotoxin affect inhibitions of short circuit current across Bombyx mori midguts.,” Proc. Natl. Acad. Sci. USA 90:9041-9045.

Crea et al., Proc. Nat'l Acad. Sci. USA 75:5765 (1978).

Crickmore, N., D. R. Zeigler, J. Feitelson, E. Schnepf, J. Van Rie, D. Lereclus, J. Baum, and D. H. Dean (1998) “Revision of the nomenclature of the Bacillus thuringiensis pesticidal crystal proteins,” Microbiology and Molecular Biology Reviews 62:807-813.

Davis et al. Microbiology, Harper & Row, New York, (1980), pages 237, 245-47, and 274

Dean, D. H., F. Rajamohan, M. K. Lee, S. J. Wu, X. J. Chen, E. Alcantara, and S. R. Hussain (1996) “Probing the mechanism of action of Bacillus thuringiensis insecticidal proteins by site-directed mutagenesis—A minireview,” Gene 179:111-117.

De Maagd, R. A., H. van der Kleij, P. Bakker, W. J. Stiekema, and D. Bosch (1996) “Different domains of Bacillus thuringiensis d-endotoxins can bind to insect midgut membrane proteins on ligand blots,” App. Environ. Microbiol. 62:2753-2757.

English, L. and Readdy, T. L. (1989) “Delta endotoxin inhibits a phosphatase in midgut epithelial membranes of Heliothis virescens,” Insect Biochem. 19:145-152.

Estruch J J, Carozzi N B, Desai N, Duck N B, Warren G W, Koziel M G. (1997) “Transgenic plants An emerging approach to pest control,” Nature Biotechnology 15:137-141.

Feitelson, J. S., J. Payne, and L. Kim (1992) “Bacillus thuringiensis Insects and beyond,” Biotechnology 10:271-275.

Garczynski, S. F., Crim, J. W., and Adang, M. J. (1991) “Identification of putative insect brush border membrane-binding molecules specific to Bacillus thuringiensis d-endotoxin by protein blot analysis,” Appl. Environ. Microbiol. 57:2816-2820.

Garczynski, S. F. and Adang, M. J. (1996) “Interactions of Bacillus thuringiensis toxins with lipids isolated from the midgut of Manduca sexta larvae,” Abstract. Society for Invertebrate Pathology 29th Annual Meeting and IIIrd International Colloquium on Bacillus thuringiensis. Cordoba, Spain Sep. 1-6, 1996.

Ge, A. Z. et al. (1989) “Location of the Bombyx mori specificty domain on a Bacillus thuringiensis Delta-endotoxin protein,” Proc. Natl. Acad. Sci USA 86:4037-4041.

Grochulski, P., L. Masson, S. Borisova, M. Pusztai-Carey, J.-L. Schwartz, R. Brousseau, and M. Cygler (1995) “Bacillus thuringiensis Cry1A(a) insecticidal toxin crystal structure and channel formation,” J. Mol. Biol. 254:447-464.

Harlow, E. and Lane, D. (1988) Antibodies a Laboratory Manual, Cold Spring Harbor Cold Spring Harbor Laboratory, pp. 1-726.

Hofmann, C., H. Vanderbruggen, H. Hofte, J. Van Rie, S. Jansens, and H. Van Mellaert. (1988) “Specificity of Bacillus thuringiensis d-endotoxins is correlated with the presence of high affinity binding sites in the brush border membrane of target insect midguts,” Proc. Natl. Acad. Sci. USA 85:7844-7848.

Hofte, H., S. Buyssens, M. Vaeck, and J. Leemans (1988) “Fusion proteins with both insecticidal and neomycin phosphotransferase II activity,” FEBS Lett. 226:364-370.

Hogrefe, H. H., J. R. Amberg, B. N. Hay, J. A. Sorge, and B. Shopes (1993) “Cloning in a bacteriophage lambda vector for the display of binding proteins on filamentous phage,” Gene 137:85-91.

Hunter, W. and Greenwood, F. (1962) “Preparation of iodine-131 labeled human growth hormone of high specific activity,” Nature 194:495-496. Keohavong, P., and Thilly, W. G. (1989) “Fidelity of DNA polymerases in DNA amplification,” Proc. Natl. Acad. Sci. U.S.A. 86:9253-9257.

Knight, P. J., N. Crickmore, and D. J. Ellar (1994) “The receptor for Bacillus thuringiensis Cry1A(c) delta-endotoxin in the brush border membrane of the lepidopteran Manduca sexta is aminopeptidase N.” Molec. Microbiol. 11:429-436.

Knowles, B. H. and J. A. T. Dow (1993) “The crystal delta-endotoxins of Bacillus thuringiensis—models for their mechanism of action on the insect gut,” Bioassays 15:469-476.

Lambert, B. and Peferoen, M. (1992) “Insecticidal promise of Bacillus thuringiensis,” Bioscience 42:112-122.

Lee, M. K., F. Rajamohan, F. Gould, and D. H. Dean (1995) “Resistance to Bacillus thuringiensis CryIA d-endotoxins in a laboratory-selected Heliothis virescens strain is related to receptor alteration,” Appl. Environ. Microbiol. 61:3836-3842.

Lee, M. K., B. A. Young and D. H. Dean (1995) “Domain III exchanges of Bacillus thuringiensis CryIA toxins affect binding to different gypsy moth midgut receptors,” Biochem. Biophys. Res. Comm. 216:306-312.

Li, J., Carroll, J., and Ellar, D. J. (1991) “Crystal structure of insecticidal d-endotoxin from Bacillus thuringiensis at 2.5 A resolution,” Nature 353:815-821.

Masson, L., Y.-J. Lu, A. Mazza, R. Brosseau, and M. J. Adang (1995) “The Cry1A(c) receptor purified from Manduca sexta displays multiple specificities,” J. Biol. Chem. 270:20309-20315.

Marzari, R., P. Edomi, R. K. Bhatjagar, S. Ahmad, A. Selvapandiyan, and A. Bradbury (1997) “Phage display of Bacillus thuringiensis Cry1A(a) toxin,” FEBS Letters 411:27-31.

Maxam et al., Meth. Enzymol., 65:499 1980

Messing et al., Nucleic Acids Res., 9:309 1981

Neumann et al., EMBO J., 1:84 1982

Parmley, S. F. and Smith, G. P. (1988) “Antibody-selectable filamentous phage vectors affinity purification of target genes,” Gene 73:305-318.

Rajamohan, F., J. A. Cotrill, F. Gould, and D. H. Dean (1996) “Role of domain II, loop 2 residues of Bacillus thuringiensis CrylAb d-endotoxin in reversable and irreversable binding to Manduca sexta and Heliothis virescens,” J. Biol. Chem. 271:2390-2396.

Raymond, M. (1985) “Presentation d'un programme d'analyse log-probit pour micro-ordinateur,” Cah. ORSTOM, Ser. Entom. Med. Parasitol. 22:117-121.

Saiki, R. K., Gelfand, D. H., Stoffel, S., Scharf, S. J., Higuchi, R., Hom, G. T., Mullis, K. B., and Erlich, H. A. (1988) “Primer-directed enzymatic amplification of DNA with a thermostable DNA polymerase,” Science 239:487-491.

Sambrook et al., Molecular Biology: A Laboratory Manual, Cold Spring Harbor Press, Cold Spring Harbor, N.Y. (1989).

Sangadala, S., F. S. Walters, L. H. English, and M. J. Adang (1994) “A mixture of Manduca sexta aminopeptidase and phosphatase enhances Bacillus thuringiensis insecticidal Cry1A(c) toxin binding and ⁸⁶Rb\K⁺ efflux in vitro,” J. Biol. Chem. 269:10088-10092.

Schmitz, J., H. Preiser, D. Maestracci, B. K. Ghosh, J. J. Cerda, and R. K. Crane (1973) “Purification of the human intestinal brush border membrane,” Biochim. Biophys. Acta 323:98-112.

Schnepf, H. E. (1995) “Bacillus thuringiensis toxins regulation, activities and structural diversity,” Curr. Opin. Biotech. 6:305-312.

Schwartz, J.-L., Y. J. Lu, P. Soehnlein, R. Brousseau, L. Masson, R. Laprade, and M. J. Adang (1997) “Ion channels formed in planar lipid bilayers by Bacillus thuringiensis toxins in the presence of Manduca sexta midgut receptors,” FEBS lett. 412:270-276.

Scott, J. K. and Smith, G. P. (1990) “Searching for peptide ligands with an epitope library,” Science 249:386-390.

Smith, G. P. (1988) “Filamentous phage assembly Morphogenetically defective mutants that do not kill the host,” Virology 167:156-165.

Smith, G. P. and D. J. Ellar (1994) “Mutagenesis of two surface exposed loops of the Bacillus thuringiensis Cry1C d-endotoxin affects insecticidal specificity,” Biochem. J. 302:611-616.

Stemmer, W. P. C. (1994a) “Rapid evolution of a protein in vitro by DNA shuffling,” Nature (London) 370:389-391.

Stemmer, W. P. C. (1994b) “DNA shuffling by random fragmentation and reassembly: In vitro recombination for molecular evolution,” Proc. Natl. Acad. Sci. U.S.A. 91:10747-10751.

Stewart, C. N., M. J. Adang, J. N. All, H. R. Boerma, G. Cardineau, D. Tucker, and W. A. Parrott (1996) “Genetic transformation, recovery, and characterization of fertile soybean transgenic for a synthetic Bacillus thuringiensis cry1Ac gene,” Plant Physiol. 112:121-129.

Tabashnik, B. E., T. Malvar, Y.-B. Liu, N. Finson, D. Boethakur, B. S. Shin, S.-H. Park, L. Masson, R. DeMaagd, and D. Bosch (1996) “Cross-resistance of diamondback moth implies altered interactions with Domain II of Bacillus thuringiensis toxins,” Appl. Environ. Microbiol. 62:2839-2844.

Thompson, M. A., H. E. Schnepf, J. S. Feitelson (1995) “Structure, function, and engineering of Bacillus thuringiensis toxins,” Genetic Engineering 17:99-117.

Van Rie, J., S. Jansens, H. Hofte, D. Degheele, and H. Van Melleart (1990) “Receptors on the brush border membrane of the insect midgut as determinants of the specificity of B. thuringiensis delta-endotoxins,” Appl. Environ. Microbiol. 56:1378-1385.

Wabiko, H. and Yasuda, E. (1995) “Bacillus thuringiensis protoxin location of toxic border and requirement of non-toxic domain for high level in vivo production of active toxin,” Microbiology 141:629-639.

Wolfersberger, M. G., X. J. Chen, and D. H. Dean (1996) “Site-directed mutations on the third domain of Bacillus thuringiensis d-endotoxin Cry1Aa affect its ability to increase the permeability of Bombyx mori midgut brush border membrane vesicles,” Appl. Environ. Microbiol. 62:279-282.

Wolfersberger, M. G., Luthy, P., Maurer, A., Parenti, P., Sacchi, V. F., Giordana, B., and Hanozet, G. M. (1987) “Preparation and partial characterization of amino acid transporting brush border membrane vesicles from the larval midgut of the cabbage butterfly (Pieris brassicae),” Comp. Biochem. Physiol. 86A: 301-308.

Wong et al., Gene, 68:193 1997

Zoller et al., Nucleic Acids Res. 10:6487-6504 (1987)

We claim:

1. A polynucleotide molecule that comprises a nucleotide sequence encoding an active toxin and a nucleotide sequence encoding a phage vector protein.

2. A nucleotide molecule of claim 1 wherein said toxin is derived from Bacillus thuringiensis.

3. The polynucleotide molecule of claim 1 wherein said phage vector protein is derived from a filamentous phage vector.

4. The polynucleotide molecule of claim 1 wherein said nucleotide sequence encoding an active toxin and said nucleotide sequence encoding a phage vector protein are expressed as a fusion protein such that a phage is formed having said active toxin displayed on the surface thereof.

5. The polynucleotide molecule of claim 1 that encodes a fusion protein as shown in FIG. 1.

6. A polypeptide molecule comprising a phage region and a toxin region wherein said polypeptide molecule is arranged to form a phage having said toxin region displayed on the surface thereof.

7. The polypeptide molecule of claim 6 wherein said toxin region is derived from Bacillus thuringiensis.

8. The polypeptide of claim 6 having an amino acid sequence as shown in FIG. 1.

9. A method of preparing active Bacillus thuringiensis toxins comprising transforming one or more cells with a polynucleotide molecule that comprises a nucleotide sequence which encodes for an active Bacillus thuringiensis toxin and a nucleotide sequence which encodes for a phage vector protein; and
growing said one or more cells under conditions such that said polynucleotide molecule is expressed, thereby forming a fusion protein having toxic activity.

10. The method of claim 9 wherein said phage vector protein is derived from a filamentous phage vector.

11. The method of claim 9 wherein said polynucleotide molecule encodes a fusion protein having an amino acid sequence as shown in FIG. 1.

12. The method of claim 9 wherein said one or more cells are prokaryotes.

13. The method of claim 13 wherein said one or more cells are of a type selected from the group consisting of E. coli strain JM109, E. coli strain JM101, E. coli K12 strain 294, E. coli strain W 3110, E. coli X1776, E. coli XL-lBlue and E. coli B.

14. The method of claim 13 wherein said one or more cells are E. coli strain JM 109.

15. A method of screening for novel Bt toxins comprising obtaining a phage display library comprising a plurality of recombinant phage having a toxin displayed on the surface thereof; and
screening said library to identify a phage clone comprising phage which bind to a toxin specific target.

16. The method of claim 15 further comprising isolating from said phage which bind to a toxin-specific target a polynucleotide molecule having a nucleotide sequence that encodes a toxin.

17. A phage clone comprising phage that comprise a polynucleotide molecule having a nucleotide sequence that encodes a toxin, wherein said phage have said toxin displayed on the surface thereof.

18. An isolated polynucleotide molecule produced by the method of claim 16.

19. One or more plant cells transformed with a polynucleotide molecule produced by the method of claim 16.
